(19) United States
(12) Patent Application Publication
(10) Pub. No.: US 2015/0037857 A1
(43) Pub. Date: Feb. 5, 2015

(54) OIL COMPOSITIONS AND METHODS OF PRODUCTION

(71) Applicant: POET Research, Inc., Sioux Falls, SD (US)

(72) Inventor: Steven G. Redford, Brandon, SD (US)

(21) Appl. No.: 14/379,428

(22) PCT Filed: Feb. 21, 2013
(86) PCT No.: PCT/US2013/027122

§ 371 Date: Aug. 18, 2014

Related U.S. Application Data
(60) Provisional application No. 61/601,659, filed on Feb. 22, 2012.

(51) Int. Cl. C11B 1/02; A23D 9/007; C12P 7/14; C11B 1/10; C10L 1/02; C12P 7/06

(52) U.S. Cl. CPC C11B 1/025; C10L 1/02; C12P 7/06; C12P 7/14; C11B 1/10; A23D 9/007
USPC 435/162; 44/400; 435/163; 426/601

(57) ABSTRACT

The present invention relates to methods of making an oil product from plant material that is used to make alcohol (e.g., ethanol) via fermentation. The methods extract oil from the plant material (e.g., corn oil from corn) before exposing to the fermented plant material to distillation temperatures so as to reduce the levels of free fatty acids and/or alcohol esters that can be generated by exposing the oil to distillation temperatures. The present invention also related to oil products made by such methods.

### PRIORITY

This application claims benefit under 35 U.S.C. §119(e) of U.S. Provisional Patent Application No. 61/601,659 filed Feb. 22, 2012 and titled “OIL COMPOSITIONS AND METHODS OF PRODUCTION”, which is incorporated herein by reference in its entirety.

### FIELD

The present invention is related to methods of making oil compositions, especially in the context of ethanol manufacturing, and also unique oil compositions made therefrom.

### BACKGROUND

The present disclosure relates to one or more oil compositions that are manufactured as a co-product of producing an alcohol such as ethanol. Ethanol can be produced from, e.g., grain-based feedstocks (e.g., corn, sorghum/milo, barley, wheat, soybeans, etc.), from sugar (e.g., sugar cane, sugar beets, etc.), and other materials derived from plant sources. In addition to the manufacture of alcohol from, e.g., carbohydrate materials of a feedstock, a number of co-products can be generated that are additional sources of revenue for the manufacturer. These co-products include, e.g., carbon dioxide gas for the industrial and food industries, protein rich animal feed products, and oils.

In a typical ethanol plant, corn, sugar cane, other grain, beets or other plants are used as a feedstock and ethanol is produced from starch contained within the corn, or other plant feedstock. In the case of a corn facility, corn kernels can be used to prepare starch-containing material for processing. Initial treatment of the feedstock can vary by feedstock type. Generally, however, the starch and sugar contained in the plant material is extracted using a combination of mechanical and chemical means.

For example, starch-containing material can be slurried with water and treated with heat to convert the starch into sugar (e.g., glucose). Many ethanol production facilities convert grain starch to sugar through a heat intensive jet-cooker step. After converting starch into sugar, the sugar can be fermented, where the sugar is converted by an ethanologen (e.g., yeast) into ethanol. The fermentation product is referred to as beer, which comprises a liquid component, including ethanol, water, and soluble components, and a solids component, including unfermented particulate matter (among other things).

In a typical ethanol plant, the fermentation product is sent to a distillation system where the fermentation product is distilled and dehydrated into ethanol. The residual matter (e.g., whole stillage) can be dried into dried distillers grains (DDG) and sold, for example, as an animal feed product. Additionally, methods of removing oil found in the stillage after distillation are known in the art.

However, when oil is removed from whole stillage after distillation the content of free fatty acids tends to be higher than desired. The percentage of free fatty acids (% FFA) can be used as a primary indicator of oil quality since the % FFA is generally considered an indication of the amount of post-processing that may be required for final use of the oil (for biodiesel, for example). Free fatty acids can be produced as a result of heating the oil found in the grain feedstock. The heating involved in many ethanol processes (e.g., converting starch to sugar, distillation, and the like) can cause oil degradation. Another indication of oil degradation due to heating is the generation of ethanol esters.

With the advent of “cold cook” ethanol production, the use of enzymes can be employed instead of excessive heat in order to convert starch in grain material to sugar. The recovery of oil from such a cold cook process is detailed in U.S. patent application Ser. No. 12/208,127 entitled “Oil Composition and Method of Recovering the Same” filed Sep. 10, 2008, which is hereby incorporated by reference in its entirety. The resulting percentage of free fatty acids derived from this cold cook process is much lower (e.g., less than 3-5%) than oils generated through a jet cooker ethanol facility, however, the oil can still be subjected to elevated heat during, e.g., the distillation step.

Methods of making oil having relatively low free fatty acid content and/or low ethyl ester content are desirable, especially as co-products of ethanol manufacturing.

### SUMMARY

The present invention provides methods of making oil compositions that are relatively low in free fatty acids and/or low in alcohol esters (e.g., ethanol esters).

The methods are in the context of making alcohol from plant material (e.g., grains such as corn kernels). The present invention appreciates that undue exposure to elevated temperatures can degrade the oil component of plant material (e.g., grains) and increase the content of one or more free fatty acids and/or one or more alcohol esters (e.g., ethanol esters).

According to the present invention, oil is separated from one or more alcohol production process streams at least prior to distillation.

Advantageously, the oil can avoid undue exposure to elevated temperatures (e.g., typical distillation temperatures) and have relatively lower amounts of one or more free fatty acids and/or one or more alcohol esters such as ethanol esters as compared to oil produced from alcohol manufacturing and that is exposed to distillation temperature(s) in one or more distillation systems.

According to one aspect of the present invention, a method of making an oil product includes providing a plant material having oil, and one or more oligosaccharides and/or one or more polysaccharides; converting at least a portion of the one or more oligosaccharides and/or one or more polysaccharides into one or more monosaccharides; fermenting at least a portion of the one or more monosaccharides to form a fermentation product comprising the oil and a biochemical; and separating at least a portion of the oil from the fermentation product to form an oil product, wherein the oil product comprises one or more free fatty acids, wherein the one or more free fatty acids are present in an amount in the range of from 0.05 to 2 percent by weight of the oil.

According to another aspect of the present invention, a method of making an oil product includes providing a plant material having oil, and one or more oligosaccharides and/or one or more polysaccharides; converting at least a portion of the one or more oligosaccharides and/or one or more polysaccharides into one or more monosaccharides; fermenting at least a portion of the one or more monosaccharides to form a fermentation product comprising the oil and a biochemical; and separating at least a portion of the oil from the fermentation product to form an oil product, wherein the at least a portion of the oil is not exposed to a temperature of 180° F. or greater throughout the method.

According to another aspect of the present invention, an oil product includes one or more free fatty acids, wherein the one or more free fatty acids are present in an amount in the range of from 0.05 to 2 percent by weight of the oil product; and one or more alcohol esters, wherein the one or more alcohol esters are present in an amount of 10 percent or less by weight of the oil product.

The present invention will be described in more detail below in the detailed description of the invention and in conjunction with the figures mentioned below.

### BRIEF DESCRIPTION OF THE DRAWINGS

FIG. 1 is a perspective view of a biorefinery comprising an ethanol production facility.

FIGS. 2A and 2B show exemplary process flow diagrams for generating ethanol in a “cold-cook” ethanol production facility.

FIGS. 3A to 3C show exemplary process flow diagrams for generating an oil product in a cold-cook ethanol production facility by removing the oil before the oil is exposed to distillation.

### DETAILED DESCRIPTION

The present invention will now be described in detail with reference to several exemplary embodiments thereof as illustrated using the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and descriptions that follow.

The disclosed embodiments relate to compositions and methods of generating an oil product. The oil product can be manufactured, e.g., at a low energy ethanol production facility as a co-product to the fermentation of grain materials such as corn materials. Preferably, the oil product is not subjected to high temperatures, as many ethanol facility oils are, such that the percentage of free fatty acids is greatly reduced.

The present invention relates to the manufacture and compositions of unique oil products through the production of alcohol (e.g., ethanol) from a feedstock such as corn grain material. The oil products (e.g., corn oil product) have one or more applications such as an animal feed supplement, industrial uses, biodiesel production, and human grade edible oil, to name a few. Oil generated through, e.g., ethanol production in this manner is unique as compared to traditional oils generated by ethanol producers due to the consistently low processing temperature and removal prior to distillation. This process protocol can help prevent any substantial formation of free fatty acids, which are often indicative of reduced oil quality.

Methods of making an oil product according to the present invention include providing a plant material that includes oil and one or more oligosaccharides and/or one or more polysaccharides. Plant materials that include oil and one or more oligosaccharides and/or one or more polysaccharides and are used in fermentation to make alcohol such as ethanol are well known. In preferred embodiments, one or more grains, are used to provide material that oil and one or more oligosaccharides and/or one or more polysaccharides. For example, corn grain includes the polysaccharide starch and corn oil.

Note that while particular reference is made to the use of corn kernels as the starting feedstock for providing a plant material, one or more other plant materials may be used alone or in combination in other embodiments. For example, soybean, or a combination of grains, may be utilized in some cases to generate alcohol such as ethanol and co-products. This may result in other novel compositions that are considered within the scope of the present disclosure. Accordingly, any of the disclosed ethanol production facilities may include modifications for the processing of other feedstocks instead, or in addition to, corn kernels. For example, soybean has a very large oil concentration and is well suited for the production of oil.

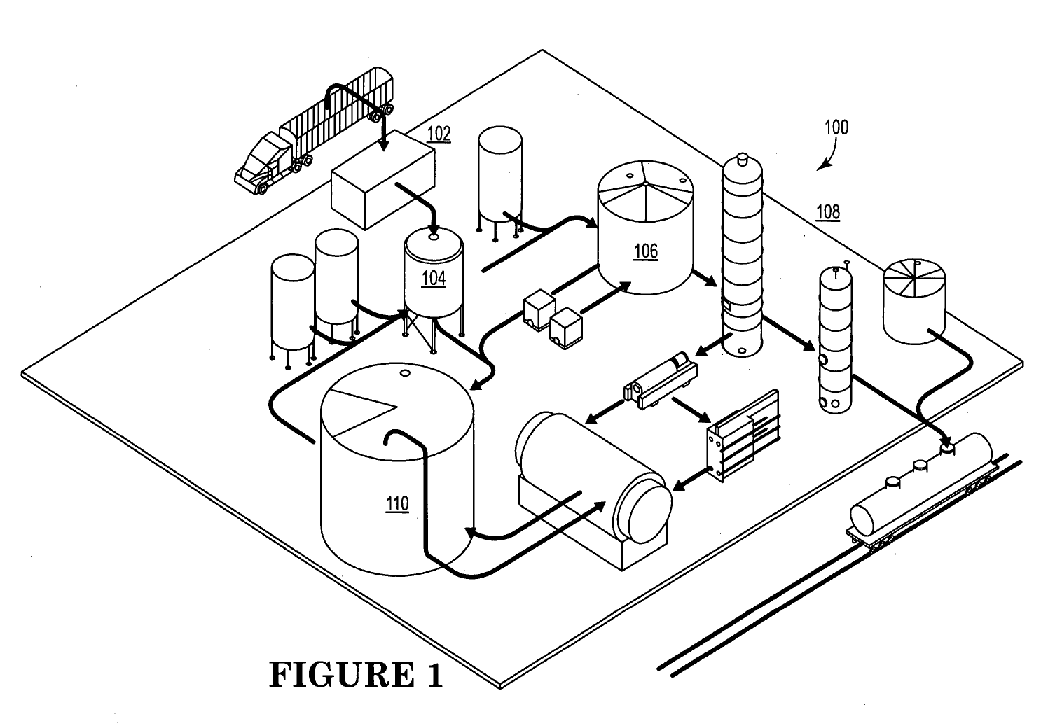

In order to facilitate disclosure, FIG. 1 is a perspective view of an exemplary biorefinery 100 comprising an ethanol production facility configured to produce ethanol from corn. The examplary biorefinery 100 includes an area 102 where corn (or other suitable material including, but not limited to, biomass, sugars, and other starch products) is delivered and prepared to be supplied to the ethanol production facility. The ethanol production facility comprises apparatus 104 for preparation and treatment (e.g., milling or fractionating) of the corn into corn flour suitable for fermentation into fermentation product in a fermentation system 106. The ethanol production facility 100 also includes a distillation system 108 in which the fermentation product can be distilled and dehydrated into ethanol. The biorefinery 100 may also include, in some embodiments, a by-product treatment system (e.g., a centrifuge, a dryer, and/or an evaporator).

As discussed below, biorefinery 100 may be a fractionation style biorefinery. However, it is considered within the scope of the present disclosure that whole kernel biorefinery plants may also be employed for the generation of oil products, as will be described in further detail below. In some embodiments, the biorefinery 100 may be referred to as a “fractionation” ethanol production facility, where the corn kernel, prior to milling, is fractionated into its three component parts. These include the outer shell (corn bran), which is predominantly a fiber material and part of the fiber component, the starch filled endosperm component, and a protein rich germ component. In some embodiments, only the endosperm component is further processed for fermentation into ethanol. In other embodiments, both the endosperm component and the germ component are further processed for fermentation into ethanol. One benefit of fractionation is that one or more low starch components can be syphoned into different process streams, thereby providing at least the high-starch endosperm to liquefaction, fermentation and distillation. This helps provide an operation that may be more efficient, lower yeast and enzyme requirements, and lower energy expended per gallon of ethanol produced. In some embodiments, one or more of the other components such as the corn bran and germ fractions may be sold as additional co products for the feed industry, or may be further processed to generate higher value co-products. Preferably, the whole corn kernel is milled and provided to the fermentation system. One benefit of milling the entire corn kernel instead of fractionating is that the germ component includes starch and oil and the endosperm component includes starch and oil so overall ethanol production can be increased due to the higher level of starch and the overall oil production can be increased due to the higher level of oil.

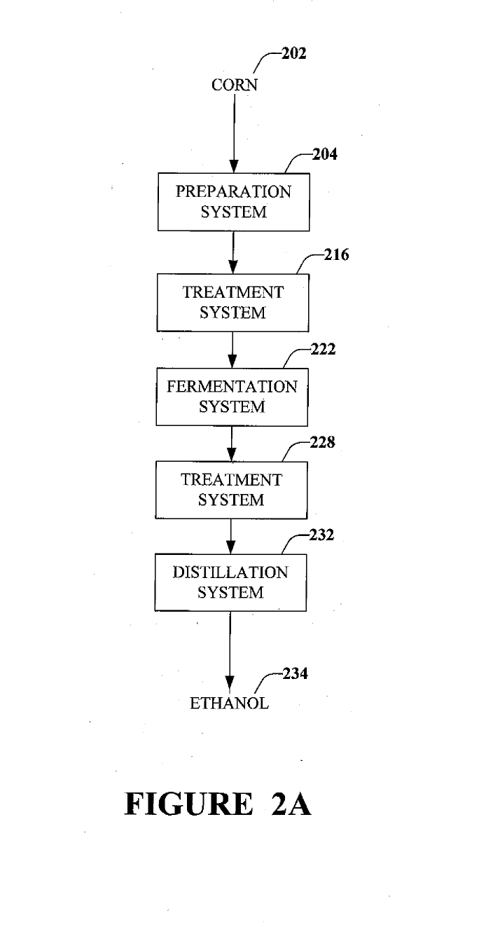

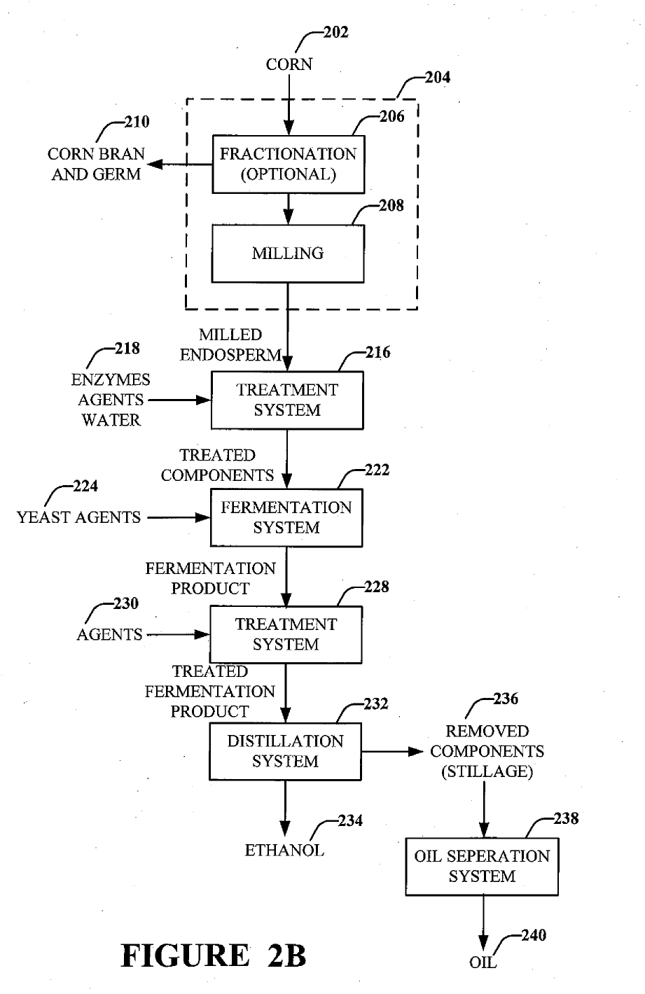

FIGS. 2A and 2B each show an exemplary process flow diagram illustrating the steps used to generate ethanol in a cold-cook ethanol production facility. In an ethanol production process, corn 202 (or other suitable feed material) may be prepared for further treatment in a preparation system 204. As shown in FIG. 2B, the preparation system 204 may include an optional fractionation system 206 to fractionate the corn kernel into its three constituents, as described above. Fractionation may employ mills, size exclusion and density separation in order to be effectual. The bran and germ components 210 can be removed for further processing or sale as raw materials. In some cases, a screening process may be performed prior or post fractionation that removes foreign material, such as rocks, dirt, sand, pieces of corn cobs and stalk, and other unfermentable material (e.g., removed components).

After fractionation, the particle size of the endosperm may be reduced by milling 208 to facilitate further processing. In processes where fractionation is omitted, the whole corn kernel may alternatively be milled to whole corn flour.

As shown in FIGS. 2A and 2B, at least a portion of the one or more oligosaccharides and/or one or more polysaccharides are convereted into one or more monosaccharides in a first treatment system 216. For example, in treatment system 216, milled corn (or endosperm) can be slurried with water, enzymes and agents 218 to liquefy the starch containing material and facilitate the conversion of starch into sugar (e.g. glucose). In many “conventional” corn-to-ethanol facilities the flour slurry is typically heated in a jet cooker in order to convert the starch into sugar. However, by using an enzymatic approach, without any external heating to convert starch to sugar, a “cold cook” process is achieved. Preferably, this cold cook conversion to monosaccarides occurs at a temperature less than 180° F., preferably less than 150° F., even more preferably less than 120° F. Such a cold cooking process can benefit from a reduction in required energy, reduced overall costs, and minimal heat damage to the starch and proteins of the corn flour. Likewise, less heat damage occurs to the fats of the corn, thereby reducing the generation of free fatty acids and ethanol esters. Of course the generation of corn meal could be performed using a conventional process involving a high heat cooking; however, this may alter the low fatty acid profile of the resulting oil due to heat damage.

As shown in FIGS. 2A and 2B, the treated plant material including one or more monosaccharides can be delivered from the treatment system 216 to the fermentation system 222, where at least a portion of the one or more monosaccharides can be fermented to form a fermentation product that includes at least the oil from the plant material and a biochemical. Such biochemicals that are formed by fermenting monosaccarides are well known and include, for example, ethanol, butanol, and the like. For example, the sugar (e.g., treated component) slurry from treatment system 216 can be converted into ethanol by an ethanologen (e.g. yeast or other agents 224) in fermentation system 222. The product of fermentation (fermentation product) is a slurry referred to as “beer,” which typically includes a liquid component, including ethanol, oil, water and soluble components, and a solids component, including unfermented particulate matter (among other things).

Optionally, the fermentation product may be treated with agents 230 in a second treatment system 228. At this stage, a low energy facility differs from a standard cold cook facility.

As shown in FIGS. 2A and 2B, a conventional ethanol process provides the entire treated fermentation product (entire solid component and liquid component) directly to a distillation system 232. In the distillation system 232, the (treated) fermentation product is distilled and dehydrated into ethanol 234.

Optionally, in some embodiments, the removed components 236 (e.g., whole stillage), which comprise water, soluble components, oil and unfermented solids (e.g., the solids component of the beer with substantially all ethanol removed), may be subjected to further processing in an oil separation system 238 to yield oil 240. This oil 240 being generated from a cold cook process has less heat damage (and subsequently lower free fatty acids) than oil from a jet cooker facility. However, despite these improvements, the oil 240 has been subjected to some heat damage in the distillation system 232. The solids of the whole stillage may be dried into dried distillers grains (DDG) in a third treatment system (not shown), where the removed components may be treated with agents and sold as an animal feed product. Other co-products, for example, syrup, may also be recovered from the stillage.

According to the present invention, at least a portion of the oil is separated from the fermentation product prior to distillation so as avoid undue exposure to distillation temperatures in distillation system 354. Typically, distillation temperatures are about 190° F. or higher. For example, 195° F. is a distillation temperature that can be used when operating under a vacuum. As another example, when operating under pressure, distillation temperatures can be from about 220-230° F. An oil product made according to the present invention can be made without exposing the oil to a temperature of 180° F. or more, preferably 150° F. or more. In many embodiments, an oil product made according to the present invention is exposed to a temperature of 120° F. or less, preferably 100° F. or less, even more preferably 90° F. or less.

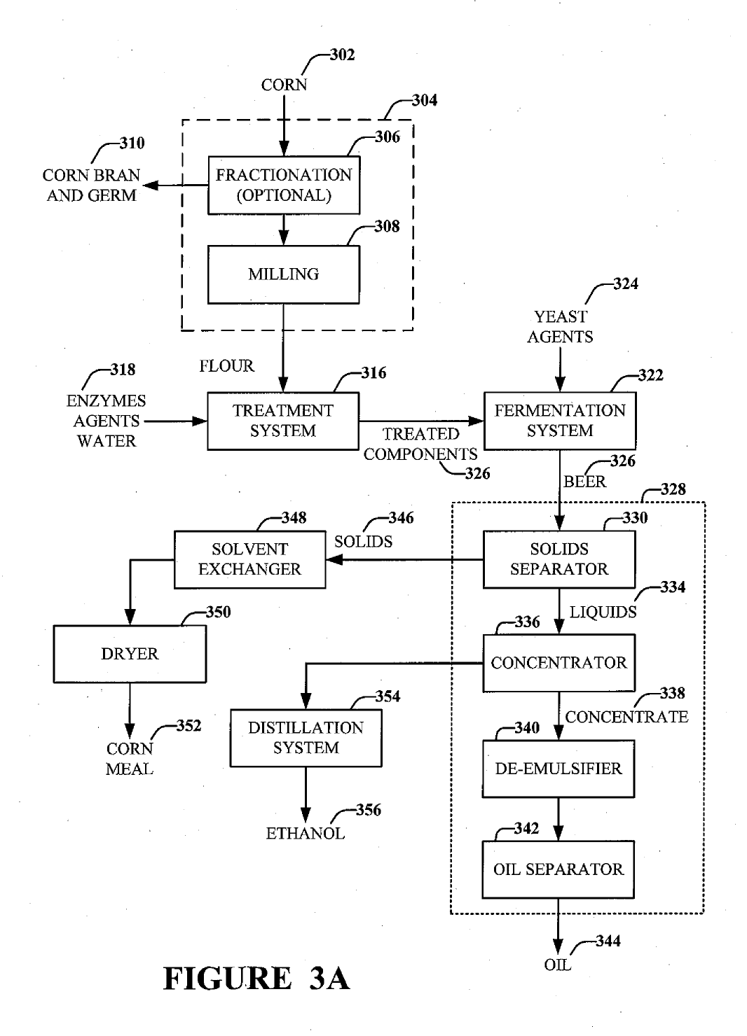

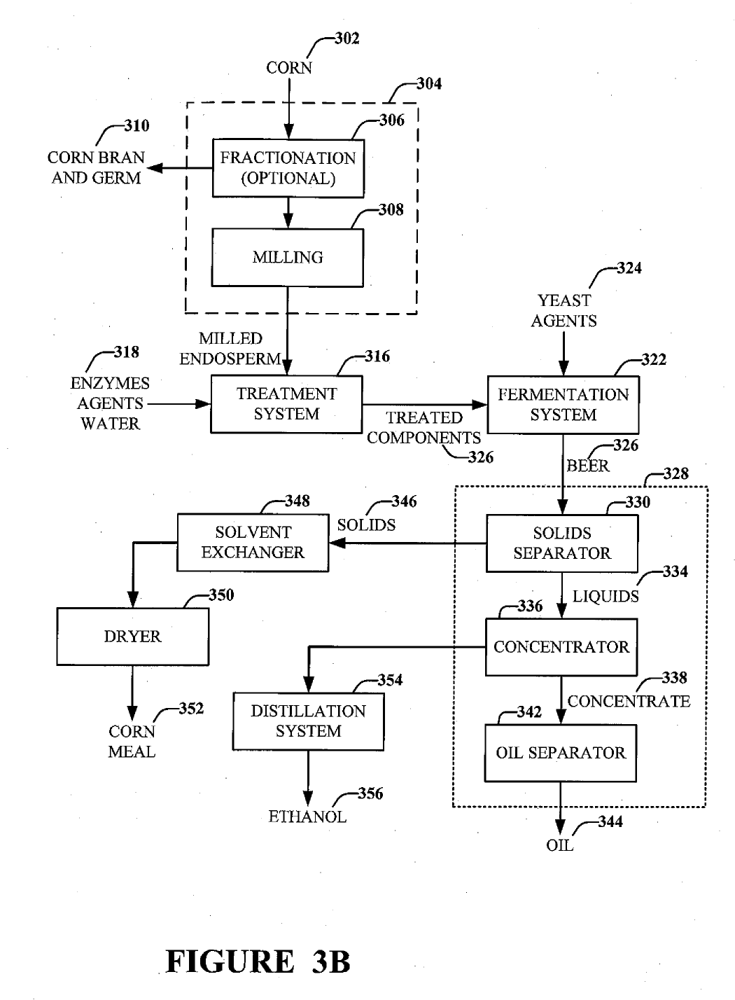

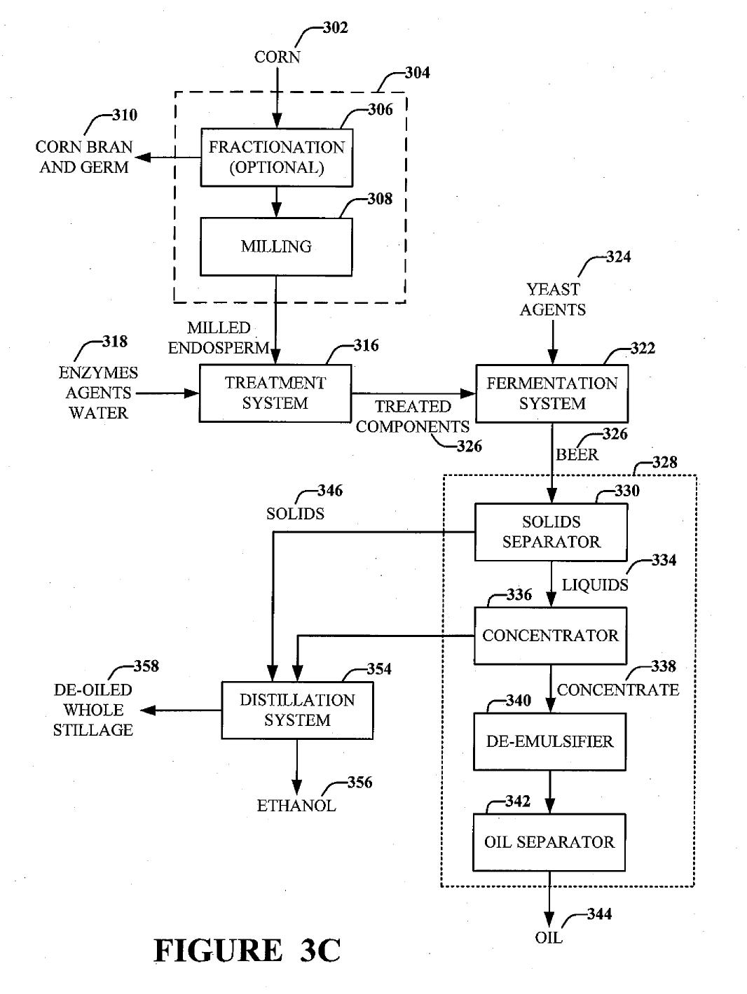

FIGS. 3A to 3C are exemplary embodiments of the present invention and show process flow diagrams illustrating the steps that can be used to generate oil in a cold-cook ethanol production facility, where the oil is not exposed to distillation. The initial stages of such a manufacturing process are similar to known cold cook plants discussed above with respect to FIGS. 2A and 2B. As discussed above, the whole corn 302 is delivered to preparation system 304 and can be milled to generate flour or, alternatively, the corn 302 can be fractionated (in a fractionation plant) using the optional fractionation system 306. The corn bran (fiber) and germ 310 components can be removed (sometimes referred to as a “full fractionation”), and the endosperm can be sent to the milling system 308 for size reduction to flour. Alternatively, only the fiber is removed and the germ component and the endosperm component can be sent to the milling system 308 for size reduction to flour (sometimes referred to as a “partial fractionation”).

As shown in FIGS. 3A to 3C, the corn material including starch can be slurried in a treatment system 316 with water and enzymes, and, optionally, one or more agents 318 to yield treated components 326 that include sugars. Yeast and other agents 324 can be added to a fermentation system 322 in order to convert the sugars to alcohol, such as ethanol, and carbon dioxide. The carbon dioxide is typically captured and sold for industrial and beverage use.

After fermentation, however, the exemplary processes shown in FIGS. 3A to 3C deviate significantly from the ethanol production practices, e.g., shown in FIGS. 2A and 2B. Since heat can damage the oil by generating an undue amount of one or more fatty acids and/or one or more ethanol esters, the resulting fermentation beer 326 is sent to an oil extraction system such as system 328 prior to any distillation so that an oil product can be extracted and not exposed to distillation temperatures. As shown in FIGS. 3A to 3C, the oil extraction system 328 preferably involves first providing the beer 326 to a solids separator 330 for removal of the solids 346 from the beer 326. This solids removal step may include any solids separator such as a screw press, centrifugation system, filter/membrane system, and/or any other known solids separator unit. Such solids separators are well known.

After separating at least a portion of the liquid component of the beer from the solids component, at least a portion of the oil may be separated from the liquid component so as to form an oil product according to the present invention. The oil may be separated from the liquid via an oil separator such as oil separator 342. Such oil separators are well known and include, e.g., a centrifuge system, a membrane based separation, and/or any other known separation methodologies for such purpose.

Optionally, the liquid component from solids separator 330 can be concentrated and/or de-emulsified before separating the oil. Deliquefying the solids can result in a liquid 334 fraction that includes water, ethanol, solubles, and an oil or oil emulsion. In a jet cooked ethanol production facility, the oil can be separated from the emulsion due to the high temperatures. However, without any such cooking step, the oil can be locked in an oil/protein emulsion layer after fermentation in a cold cook facility.

As shown in FIGS. 3A to 3C, the liquid component 334 can be concentrated via a concentrator 336 such as a centrifugation system (such as a decanter centrifuge), a filter/membrane system, and/or other known concentration techniques.

As shown in FIG. 3B, the beer 326 has the solids removed by a separator 330, the liquids 334 are concentrated in a concentrator 336, and the oil is separated out of the concentrate 338 using an oil separator 342. Concentrator 336 is able to concentrate the liquid component 334 and form concentrate 338. For example, the liquid 334 may be put under pressure against a membrane with pores that enables the ethanol, water and fines to pass through the membrane, and yet retain the larger oil or oil emulsion fraction (also referred to as a “concentrate”). The de-oiled liquids are provided to a distillation system 354 for the distillation of ethanol. Finally, the oil can be separated from the concentrate via separator 342 which can generate a clean oil product 344.

The oil extraction system 328 shown in FIG. 3B may be useful where an oil emulsion is naturally not present, or where the emulsion is able to be disrupted during and/or prior to fermentation. For example, the beer may be treated immediately post fermentation in order to disrupt emulsion by adding surfactants. Likewise, if the oil separator 342 operates with enough physical force to disrupt the surface properties of the emulsion, the oil may be separated without the need for a discrete de-emulsification step (discussed below). Further, emulsion may be absent under specific feedstock or processing conditions. Alternatively, the solids separator 330, concentrator 336 and oil separator 342 may be combined into a single piece of machinery, such as a three-phase centrifuge.

In some embodiments, the concentrate includes an emulsion and it may be desirable to optionally “break” the emulsion prior to oil separation. The de-emulsification may be performed by application of surfactant, electrical charge, or pH adjustment. As shown in FIGS. 3A and 3C, the oil/emulsion concentrate 338 is provided to a de-emulsifier 340 which may employ pH adjustment, electrostatic forces (i.e., passing the emulsion between two charged plates), physical shearing, surfactant addition, and other chemical treatments which change the surface properties of the emulsion. After the emulsion is disrupted, the concentrate may be supplied to a final oil separator 342 which generates a clean oil product 344. This separator may again include a centrifuge system or a membrane based separation. The oil product obtained by a method according to the present invention can have much lower levels of free fatty acids and ethanol esters compared to oils generated from a jet cooker ethanol facility, or even a cold cook ethanol facility where oil is collected post distillation.

Free fatty acid levels of oil products according to the present invention can be less than 10 percent by weight of the oil product, preferably 2 percent or less, even more preferably 1.8 percent or less, and even more preferably 1.6 percent or less by weight of the oil product. Often, the free fatty acid level of the oil products is greater than 0.05 percent by weight of the oil product, even 0.1 percent or greater, or even 0.4 percent or greater.

Free fatty acids that can be generated by heating oil derived from plant material are well known. Examples include, e.g., caproic acid, capric acid, caprylic acid, lauric acid, myristic acid, palmitic acid, stearic acid, oleic acid, linolenic acid, linoleic acid, arachidic acid, and mixtures thereof.

Ethanol ester levels of oil products according to the present invention can be less than 20 percent by weight of the oil product, preferably 10 percent or less, even more preferably 7 percent or less, and even more preferably 5 percent or less by weight of the oil product. Often, the ethanol ester level of an oil product is greater than 0.05 percent by weight of the oil product, even 0.1 percent or greater, or even 0.5 percent or greater.

Ethanol esters that can be generated by heating oil derived from plant material are well known. Examples include esterification of any of the fatty acids described herein with ethanol.

Optionally, the oil extraction system 328 may be combined with other low energy solutions employed pre-distillation to improve efficiency. For example, the solids 346 separated from the beer 326 may optionally be provided to a solvent exchange system 348 in order to replace water contained in the solids with a volatile solvent.

Water has a very high boiling point, heat capacity, and heat of vaporization. Because of these characteristics, a large amount of energy is often required to heat water to a temperature sufficient to vaporize the water and then carry out the vaporization. The solvent exchanger 348 is designed to wash or extract water from wet solids using a solvent with a low heat of vaporization, heat capacity, and boiling point, or some combination of these three characteristics. After some water has been extracted from the wet solids, the wet solids, which now include a quantity of the solvent, are dried using lower amounts of energy in a dryer 350. Because of the availability of ethanol in an ethanol plant, ethanol is a particularly relevant solvent to be used in the wash, or water extraction, process of the present inventions. Ethanol is known to have a heat capacity of about 0.58 Btu/lb-F, which is approximately half of heat capacity of water. The boiling point of ethanol is approximately 173 degrees Fahrenheit versus the boiling point of water, which is approximately 212 degrees Fahrenheit. Finally, the heat of vaporization of ethanol is approximately 362 Btu/lb versus the heat of vaporization of water, which is approximately 980 Btu/lb.

The concentration of ethanol in wet solids is determined by the fermentation process, but is typically between 11 and 20 percent, although concentrations of amounts lower than this range and higher than this range are compatible with the embodiments disclosed in this application. This range of typical ethanol concentrations of wet solids separated from fermented beer is relatively low compared to the water concentration in the wet solids. Wet solids separated from fermented beer have a relatively high water content and a relatively low ethanol content.

For the solvent exchange, the solid may have a volume of liquid washed through it without the concentration of liquid in the solids changing throughout the wash. Alternatively, the solids may be diluted in the solvent and then deliquefied again. Vat based systems, filter belt systems, single solvent exchange systems, multiple solvent exchanges, and countercurrent washing systems are all considered within the scope of the solvent exchanger 348.

The solids from this solvent exchange are provided to a dryer 350 to generate the corn meal 352, which can help capitalize on pre-distillation processing. The solvent exchange replaces the residual water in the solids with a solvent (typically ethanol) with a lower heat of vaporization. As such, the dryer may be operated at a much lower temperature as compared to dryers that are utilized in many conventional plants. In some embodiments, the dryers 350 operate at or below about 150° F.

By directing only the fermented liquid mixture to the distillation system 354, the distillation system 354 is less susceptible to fouling, which is often primarily caused by solids present when fermented beer is distilled directly. With a reduced susceptibility to fouling, complicated anti-fouling provisions of the distillation system may be unnecessary, thereby reducing the complexity and cost of the distillation system. Because the fermented liquid mixture processed by the distillation system can be substantially free of solid components and oils, heat energy applied to the distillation system will typically only have to heat those minimal solids dissolved in or otherwise present in the fermented liquid mixture. This can reduce distillation system heat energy requirements as compared to a distillation system that must heat both the solid and liquid components of fermented beer.

In some specific embodiments, only one cycle of dilution and deliquefaction is performed. In alternate embodiments, it may be desirable to undergo multiple dilutions and deliquefaction steps in order to replace as much of the water in the solids with ethanol (or other solvent) as economically possible. In these multiple solvent exchange cycles, the liquid from a later stage may be recycled as the dilution liquid for the previous cycle in a counter-current methodology in order to more efficiently replace the water with solvent. Additionally, in some embodiments, it is desirable to remove some protein fractions prior to the solvent exchange. In these embodiments, during the first dilution, a specific solvent type and concentration may be employed to solubilize desired proteins. These proteins may then be recovered from the solvent by changing concentration or temperature. One such protein in corn that may be removed is zein. Although zein is a protein, many animals do not easily digest it, and the corn meal generated can benefit from its removal. Further, zein has a number of industrial and human consumption end uses that make it a valuable co-product in its own right.

Note that FIGS. 3A and 3B both benefit from a system where the beer is being separated into a liquid and solids stream for the purposes of low energy drying of the solids. This synergy capitalizes on sharing the initial separation step. However, as illustrated in FIG. 3C, it is considered within the scope of some embodiments that oil separation from beer occurs independent from any low-energy drying processes. In this example illustration, the solids 346 that are separated from the beer 326 are reintroduced to the liquid stream coming off of the concentrator 336 during, or prior to, distillation. This results in a whole stillage 358 that is very similar to many conventional whole stillages with the notable exception that it has a reduced oil content.

The embodiments as disclosed and described in the application (including the Figures and Examples) are intended to be illustrative and explanatory of the present invention. Modifications and variations of the disclosed embodiments, for example, of the apparatus and processes employed (or to be employed) as well as of the compositions and treatments used (or to be used), are possible; all such modifications and variations are intended to be within the scope of the present invention.

The word “exemplary” is used to mean serving as an example, instance, or illustration. Any embodiment or design described as “exemplary” is not necessarily to be construed as preferred or advantageous over other embodiments or designs, nor is it meant to preclude equivalent exemplary structures and techniques known to those of ordinary skill in the art. Rather, use of the word exemplary is intended to present concepts in a concrete fashion, and the disclosed subject matter is not limited by such examples.

The term “or” is intended to mean an inclusive “or” rather than an exclusive “or.” To the extent that the terms “comprises,” “has,” “contains,” and other similar words are used in either the detailed description or the claims, for the avoidance of doubt, such terms are intended to be inclusive in a manner similar to the term “comprising” as an open transition word without precluding any additional or other elements.

Note that the various features of the present invention described above may be practiced alone or in combination.

1. A method of making an oil product comprising:
providing a plant material comprising:
oil; and
one or more oligosaccharides and/or one or more polysaccharides;
converting at least a portion of the one or more oligosaccharides and/or one or more polysaccharides into one or more monosaccharides;
fermenting at least a portion of the one or more monosaccharides to form a fermentation product comprising the oil and a biochemical; and
separating at least a portion of the oil from the fermentation product to form an oil product, wherein the oil product comprises one or more free fatty acids, wherein the one or more free fatty acids are present in an amount in the range of from 0.05 to 2 percent by weight of the oil.

2. The method of claim 1, further comprising, after the separating step, distilling at least a portion of the fermentation product.

3. (canceled)

4. The method of claim 1, wherein the at least a portion of the oil is not exposed to a temperature of 180° F. or greater throughout the method.

5. The method of claim 1, wherein the at least a portion of the oil is not exposed to a temperature for distilling ethanol from the fermentation product throughout the method.

6. The method of claim 1, wherein the plant material comprises grain material.

7. The method of claim 1, wherein the biochemical comprises ethanol.

8. (canceled)

9. (canceled)

10. The method of claim 1, wherein providing a plant material comprises fractionating a plurality of corn kernels to provide a grain material, wherein the plurality of corn kernels comprise a fiber component, an endosperm component, and a germ component, and wherein the grain material comprises at least the endosperm component.

11. (canceled)

12. (canceled)

13. The method of claim 1, where the converting comprises exposing at least a portion of the one or more oligosaccharides and/or one or more polysaccharides to one or more enzymes to convert the one or more oligosaccharides and/or one or more polysaccharides into one or more monosaccharides.

14. The method of claim 1, wherein the plant material comprises corn kernel material comprising starch and wherein converting at least a portion of the one or more oligosaccharides and/or one or more polysaccharides into one or more monosaccharides comprises converting at least a portion of the starch to sugar.

15. The method of claim 1, wherein the fermenting forms a fermentation product comprising a solid component and a liquid component, wherein the liquid component comprises at least a portion of the oil, and further comprising separating at least a portion of the liquid component from the solid component.

16. The method of claim 15, further comprising concentrating at least a portion of the liquid component into a concentrate comprising the oil and separating at least a portion of the oil from the concentrate.

17. The method of claim 16, further comprising de-emulsifying the concentrate prior to separating at least a portion of the oil from the concentrate.

18-20. (canceled)

21. The method of claim 1, wherein the oil product comprises one or more alcohol esters, wherein the one or more alcohol esters are present in an amount of 10 percent or less by weight of the oil product.

22. (canceled)

23. A method of making an oil product comprising:
providing a plant material comprising:
oil; and
one or more oligosaccharides and/or one or more polysaccharides;
converting at least a portion of the one or more oligosaccharides and/or one or more polysaccharides into one or more monosaccharides;
fermenting at least a portion of the one or more monosaccharides to form a fermentation product comprising the oil and a biochemical; and
separating at least a portion of the oil from the fermentation product to form an oil product, wherein the at least a portion of the oil is not exposed to a temperature of 180° F. or greater throughout the method.

24-27. (canceled)

28. An oil product comprising:
one or more free fatty acids, wherein the one or more free fatty acids are present in an amount in the range of from 0.05 to 2 percent by weight of the oil product; and
one or more alcohol esters, wherein the one or more alcohol esters are present in an amount of 10 percent or less by weight of the oil product.

29. The oil product of claim 28, wherein the one or more alcohol esters are present in an amount of 7 percent or less by weight of the oil product.

30. The oil product of claim 28, wherein the one or more alcohol esters are present in an amount of 5 percent or less by weight of the oil product.

31. The oil product of claim 28, wherein the one or more alcohol esters comprise one or more ethanol esters.

32. The oil product of claim 28, wherein the one or more free fatty acids are present in an amount in the range of from 0.1 to 1.8 percent by weight of the oil product.

33. The oil product of claim 28, wherein the one or more free fatty acids are present in an amount in the range of from 0.4 to 1.6 percent by weight of the oil product.
